(19) United States
(12) Patent Application Publication
(10) Pub. No.: US 2009/0299415 A1
(43) Pub. Date: Dec. 3, 2009

(54) PEDICULAR PERCUTANEOUS MINIMALLY INVASIVE SCREW

(75) Inventor: Luis Henrique Mattos Pimenta, Sao Paulo (BR)

Correspondence Address: SCULLY SCOTT MURPHY & PRESSER, PC, 400 GARDEN CITY PLAZA, SUITE 300, GARDEN CITY, NY, 11530, US

(73) Assignees: Luis Henrique Mattos Pimenta, Sao Paulo (BR); MDT Industria e Comercio de Implantes Ortopedicos Ltda, Rio Claro (BR)

(21) Appl. No.: 12/351,936

(22) Filed: Jan. 12, 2009

(30) Foreign Application Priority Data
May 29, 2008 (BR) PI 0801980-0

(51) Int. Cl. A61B 17/86; A61B 17/70

(52) U.S. Cl. 606/305; 606/301; 606/264

(57) ABSTRACT

A pedicular percutaneous minimally invasive screw for use in connection with progressive cannulae which allows screws to be installed in a patient without necessarily cutting or destroying ligaments and muscles, the progressive cannulae going from larger to smaller diameters until the point where a perforation is made, through which the pedicular percutaneous screw enters, guided by a fine filament which precisely positions the screw in the location desired. Adjacent screws are connected via a connection bar that unites the two screws and fixes the bones of a patient's column, wherein the connecting bar is attached to the screw by a compression screw, which is threaded on the body itself.

### FIELD OF THE INVENTION

The present invention is in the field of surgical tools.

### BACKGROUND OF THE INVENTION

Conventional percutaneous screws currently available on the market require a large entrance wound resulting in much longer recuperation time along with possible post-surgical complications, and great discomfort, such as: intra-muscular bleeding, dermal scarring and associated pain. Lesions to the muscles, ligaments and skin may also occur, thus causing an even more difficult post-surgery recuperation requiring longer resting period for the patient.

What is needed therefore is a pedicular screw which is minimally invasive and results in the briefest postoperative recovery period as possible with mininimal side effects.

### SUMMARY OF THE INVENTION

The present invention relates to a pedicular percutaneous minimally invasive screw for use in surgical procedures involving the thoracic/lumbar column. Examples of pathologies involving the thoracic/lumbar column are degenerative illnesses of the disk, trauma of the thoracic/lumbar column, spondylosis and general deformities at sole or multiple levels of the column. The present invention permits a screw to be placed thorough the skin using progressive cannulae mechanisms guided by radiographic image and anchored precisely and very safely directly into the bone resulting in minimally invasive incisions. This results in preserving the tissues, muscles and ligaments, less recuperation time and postoperative pain for the subject patient. The present pedicular percutaneous minimally invasive screw is designed to inflict the least possible amount of trauma on the subject patient, particularly through the use of the aforementioned progressive cannulae which allows muscles and ligaments to be avoided during incision. The cannulae, as the name implies, are progressive, ranging from larger to smaller diameters. Cannula are progressively used to locate a pathway, which does not interfere with any ligaments or muscles where the screw can be precisely positioned in the desired location, guided by a fine filament.

Since the surgeon cannot operate by sight due to the extremely small incisions involved, a system of radiographic images is used, which allows the surgery to be completed with the highest precision possible.

The cannulae have extensions beyond the surface of the subject patient's skin, which do not increase the incision or the aggression, yet provide the surgeon with a good notion of how far to lower the bar which will unite the two screws to fix the bones of the patient's column. For each one of these screw placement stages there is a series of specific mechanisms which give the system added safety, speed, rigidity and versatility. These properties differentiate the pedicular percutaneous screw system from the other available prior art systems

### BRIEF DESCRIPTION OF THE DRAWINGS

FIG. 1 is a section view in a side elevation of the pedicular percutaneous minimally invasive screw, showing the indication of detail A.

FIG. 2 is a view in front elevation of the pedicular percutaneous minimally invasive screw.

FIG. 3 is a view in side elevation showing detail “A” of the pedicular percutaneous minimally invasive screw's thread.

FIG. 4 is a view in side elevation of the pedicular percutaneous minimally invasive screw's polyaxial ring.

FIG. 5 is a view in front elevation of the pedicular percutaneous minimally invasive screw's polyaxial ring.

FIG. 6 is a plan view of the pedicular percutaneous minimally invasive screw's polyaxial cover.

FIG. 7 is a section view of the side elevation of the pedicular percutaneous minimally invasive screw's polyaxial cover.

FIG. 8 is the system of pedicular percutaneous minimally invasive screw in front view.

FIG. 9 is a section view showing the assembly of the components which form the pedicular percutaneous minimally invasive screw.

FIG. 10 is a plan view showing the assembly of the components which form the pedicular percutaneous minimally invasive screw.

FIG. 11 is a front view showing the assembly of the components which form the pedicular percutaneous minimally invasive screw.

FIG. 12 is a side view showing the assembly of the components which form the pedicular percutaneous minimally invasive screw.

FIG. 13 is a side elevation view of the connecting bar of the pedicular percutaneous minimally invasive screw.

FIG. 14 is a front view of the connecting bar of the pedicular percutaneous minimally invasive screw.

FIG. 15 is a perspective view of the connecting bar of the pedicular percutaneous minimally invasive screw.

FIG. 16 is a front view of the compression screw, showing section B-B of the pedicular percutaneous minimally invasive screw.

FIG. 17 is a section view B-B of the compression screw of the pedicular percutaneous minimally invasive screw.

FIG. 18 is a detailed view of the compression screw of the pedicular percutaneous minimally invasive screw.

FIG. 19 is a view in perspective of the compression screw of the pedicular percutaneous minimally invasive screw.

FIG. 20 is a view in side elevation of filament which guides the pedicular percutaneous minimally invasive screw.

FIG. 21 is a view in perspective of the filament which guides the pedicular percutaneous minimally invasive screw.

FIG. 22 is a view in perspective of the components in the assembly of the pedicular percutaneous minimally invasive screw.

FIG. 23 is a general view of the application of the pedicular percutaneous minimally invasive screw in a column surgery.

### DETAILED DESCRIPTION OF THE INVENTION

The invention will be described for the purposes of illustration only in connection with certain embodiments; however, it is to be understood that other objects and advantages of the present invention will be made apparent by the following description of the drawings according to the present invention. While a preferred embodiment is disclosed, this is not intended to be limiting. Rather, the general principles set forth herein are considered to be merely illustrative of the scope of the present invention and it is to be further understood that numerous changes may be made without straying from the scope of the present invention.

The pedicular percutaneous minimally invasive screw is constituted of an assembly of components, which fit each other, forming the screw itself. The preferential material to manufacture the screw and its other components is titanium, however other materials are also contemplated, providing that the chosen material does not hamper the patient's physical integrity.

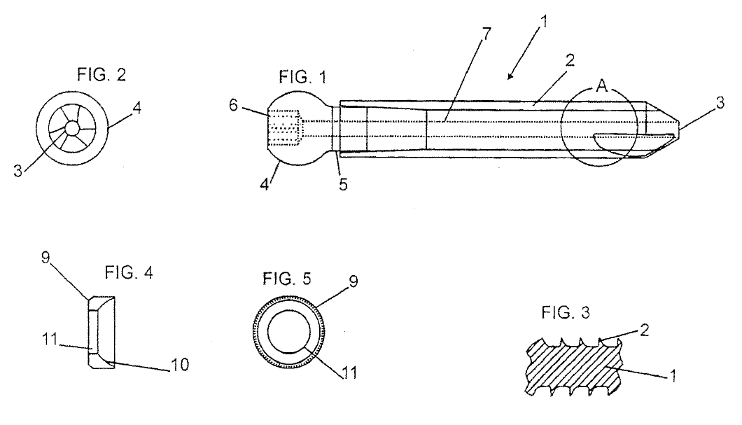

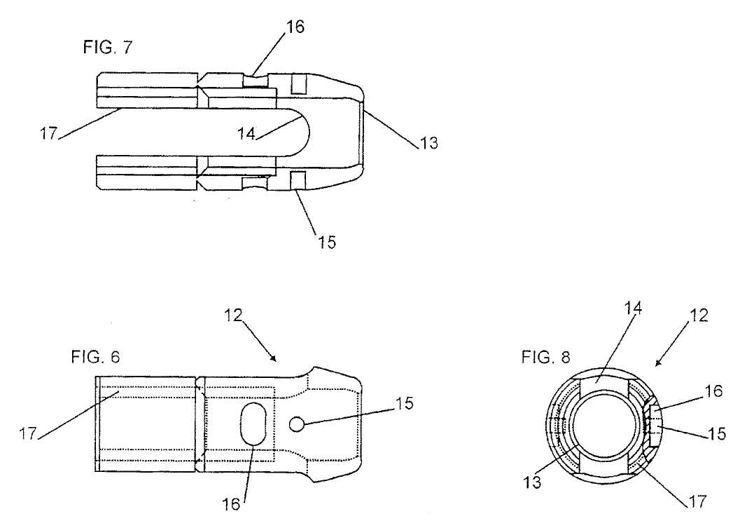

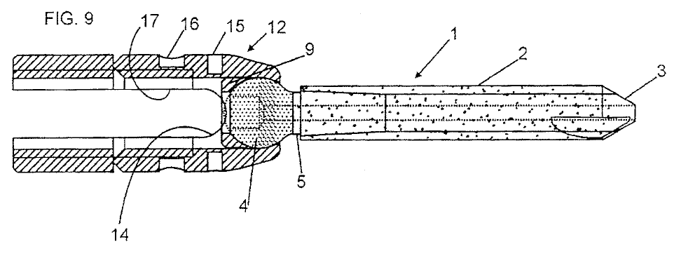

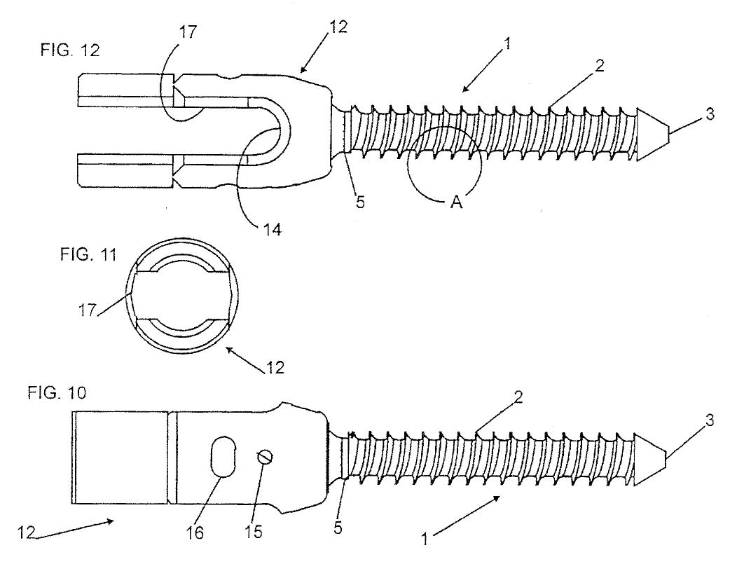

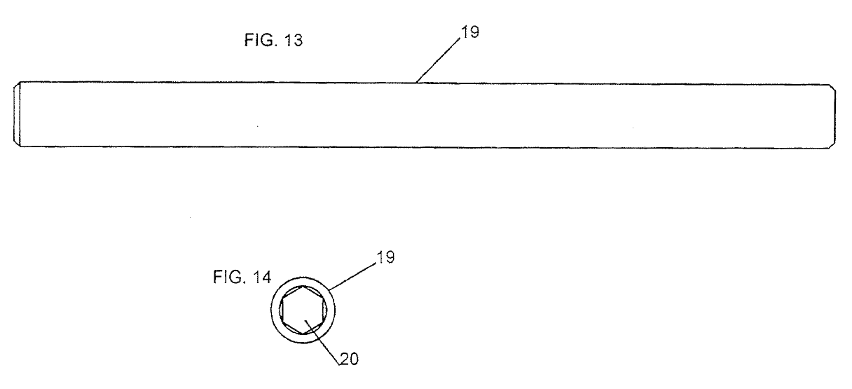

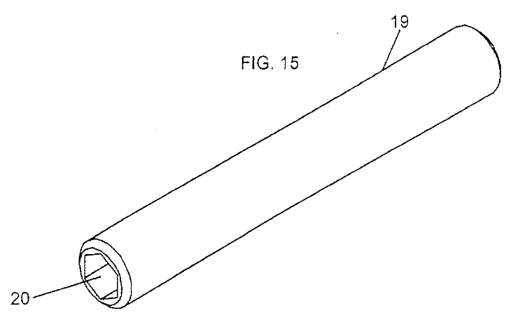

As it can be seen from FIGS. 1 to 15, the pedicular screw (1), has a parallel cylindrical body, containing a thread along it, with a profile is similar to saw blade teeth (2); this considerably helps the screw penetration without causing damages to the ligaments or muscles of the patient. The lower end is tapered to a “V-shape”, containing a sectioned edge with no point (3). The head (4) has a rounded form, having a small parallel cylindrical neck at the end of the thread (5).

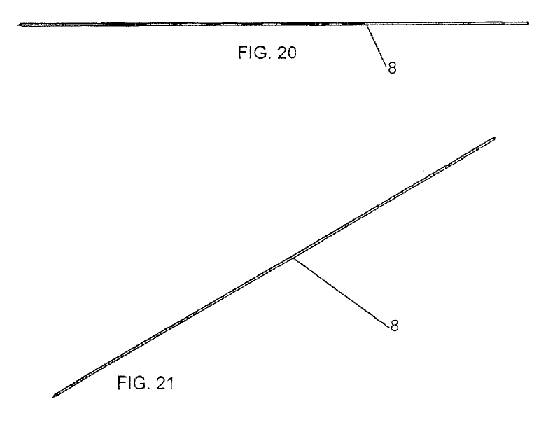

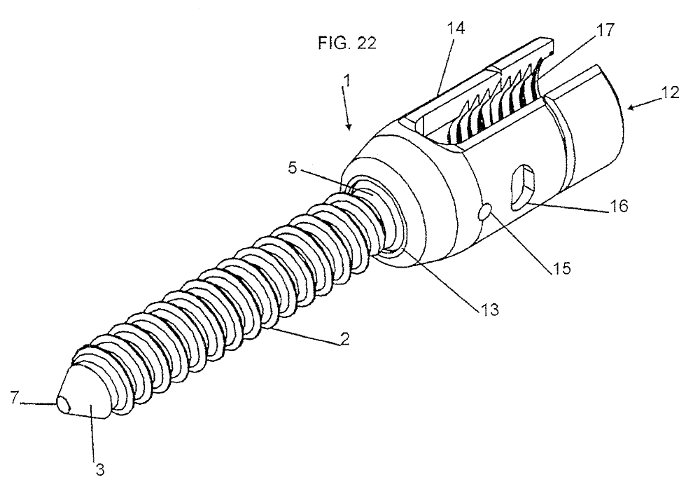

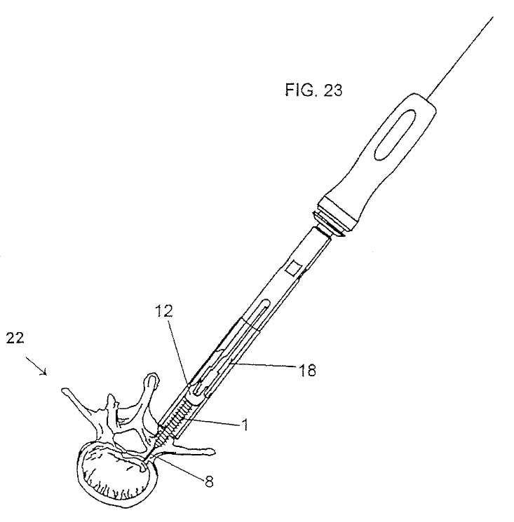

There is also a larger diameter octagonal central perforation in the screw head (6) followed by a smaller diameter parallel cylindrical perforation (7) which accompanies the entire extension of the body till the end. This perforation serves as a guide for a quite fine filament (8) (See FIGS. 20, 21 and 23, which will indicate where the pedicular percutaneous screw will be fixed onto the bone.

The second component is a polyaxial ring (9), with an external cylindrical format with rounded corners, having on one side an angular cut (10), where the screw head fits (4) and on the other side, a parallel cylindrical perforation (11).

Another component is the polyaxial cover (12). The cover can be of shape similar to a tulip and has a perforation going entirely through it (13). The pedicular screw is introduced into perforation (13) until the head (4) is fitted to the internal surface, which has the same rounded design, fitting with complete perfection, forming a sole assembly. The cover (12) contains a transversal projection in “U-shape” (14), which goes through part of the cover.

The external cylindrical body of the cover (12) has small round perforations (15) partially through the cover thickness, adjacent to the portion where the head (4) is fitted to the internal surface and an oblong, perforation (16) of a diameter larger than the diameter of perforations (15), and adjacent to perforations (15). On the internal portion of the cover there is a thread (17) responsible for the connection of the progressive cannulae (18) ranging from larger to smaller diameters. The thread (17) and the cannulae (18) will form the system of the pedicular percutaneous screw and the cannulae will enable the screw (1) to be precisely positioned in the desired location. The transversal projection in “U-shape” (14) of the cover is used to introduce the connecting bar (19) of the screw, which will support the fusion of the bone structure during surgery and after it is concluded.

The connection bar (19) is a cylindrical part with variable lengths. The use of the connecting bar will be adjusted and in accordance with the need of each situation for each patient. The diameter has only one permanent dimension. On the both ends of the bar there is a small hexagonal-shape perforation (20) of a predetermined depth.

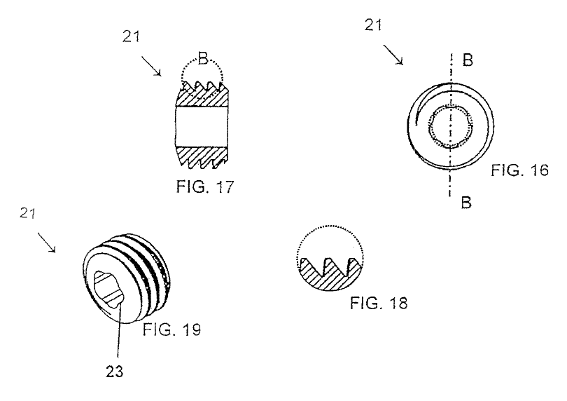

The connection bar (19) in the polyaxial cover (12) is fixed by a system compression screw (21), which is threaded onto the target body (22) and has a cylindrical format, where there is an internal octagonal perforation through (23), as it can be seen from FIGS. 16 to 23.

The progressive cannulae (18) are threaded in the internal thread of the cover (12) and they can be of different sizes, to adjust to specific needs when the surgery is performed.

The pedicular percutaneous screw (1) is treated with plasma spray material (not shown), which much facilitate the adhesion to the patient's bone, preventing rejection.

Innumerous instruments are integrally used with the system of the pedicular percutaneous screw, to install or remove the minimally invasive screw of the present invention. Such instruments are: Cannulated quick coupling star wrench; Cannulated pedicular male thread tool; Compressor pincer; Straight groove bar pincer; Hexagonal quick coupling handle; Extractor of the introduction cover; Cleaning filament; Graduated guide filament; Spondyl breaking pliers; Support for the introduction cover; Dilator; Screw length measuring device; Bar length measuring device; Long initiating needle; Cannulated probe; Bar lowerer; Introduction cover; Anti-torque cover; Manual narrow bender for bars; Soft parts protector; and Quick coupling “T” cable.

Having thus described the invention, what is claimed as new and secured by Letters Patent is:

1. A pedicular percutaneous minimally invasive screw for use in connection with progressive cannulae, said screw comprising:
(a) a cylindrical screw body having:
a first end having a tapered shaped section with a sectioned edge;
a second end having a substantially rounded head;
a continuous set of threads of saw tooth shaped profiles, adjacent to the first end;
a cylindrical neck between the head and the threads;
a first octagonal perforation located axially in the outer surface of the head; and
a second octagonal perforation of a diameter smaller that the first octagonal perforation, extending axially from the first octagonal perforation throughout the entire length of the screw body and is adapted for guiding a fine filament.

2. The pedicular percutaneous minimally invasive screw for use in connection with progressive cannulae of claim 1, further comprising:
(b) a polyaxial thrust ring with rounded corners, having a first end and a second opposite end, the first end having an axially angular cut constructed and arranged to receive the screw head; and the second end having an axially cylindrical hole constructed and arranged to receive the screw body.

3. The pedicular percutaneous minimally invasive screw for use in connection with progressive cannulae of claim 2, further comprising:
(c) a polyaxial cover substantially tulip-shaped constructed and arranged to receive the screw head, having:
a first end and a second end;
a transversal U-shape projection extending axially through the first end and through a portion of the cover and constructed and arranged to receive a connecting bar,
a bore extending axially through the cover from the first end to the second end, having an internal surface constructed and arranged to have the same surface shape of the screw head, the bore receiving the screw head;
round perforations extending radially through the cover and partially through the cover thickness, adjacent to the second end;
oblong perforations adjacent and parallel to the round perforations, extending radially through the cover;
an internal thread extending axially and partially into the cover from the first end, constructed and arranged to connect the progressive canullae to the polyaxial cover;
wherein the thread and the cannulae form the system of the pedicular percutaneous screw enabling the screw to be precisely positioned in the desired location.

4. The pedicular percutaneous minimally invasive screw for use in connection with progressive cannulae of claim 3, further comprising:
(d) a connection bar having:
a cylindrical element of adjustable lengths, having a first end and a second end, the length adjusted to correspond with the distance between adjacently installed screws; and
a hexagonal-shape perforation of a predetermined depth, at the first and second ends extending axially into said cylindrical element from the first and second ends, wherein the connecting bar is adapted to span the distance created between adjacently installed screws.

5. The pedicular percutaneous minimally invasive screw for use in connection with progressive cannulae of claim 4, further comprising:
(e) a compression screw having a cylindrical format and an internal octagonal perforation, threaded onto a target, and fixing the connection bar to the polyaxial cover.

6. The pedicular percutaneous minimally invasive screw for use in connection with progressive cannulae of claim 5, wherein the progressive cannulae are threaded in the internal thread of the cover, and are of different sizes, adjusting to specific needs when the surgery is performed.

7. The pedicular percutaneous minimally invasive screw for use in connection with progressive cannulae of claim 6, wherein the components are treated with a plasma spray coating, facilitating adhesion with a patient's bone structure and minimizing rejection.

8. The pedicular percutaneous minimally invasive screw for use in connection with progressive cannulae of claim 6, wherein the instruments used to install and remove said minimally invasive screw are selected from a group consisting of: cannulated quick coupling star wrench; cannulated pedicular male thread tool; compressor pincer; straight groove bar pincer; hexagonal quick coupling handle; extractor of the introduction cover; cleaning filament; graduated guide filament; spondyl breaking pliers; support for the introduction cover; dilator; screw length measuring device; bar length measuring device; long initiating needle; cannulated probe; bar lowerer; introduction cover; anti-torque cover; manual narrow bender for bars; soft parts protector; and quick coupling “T” cable.
